(19) United States
(12) Patent Application Publication
(10) Pub. No.: US 2010/0291221 A1
(43) Pub. Date: Nov. 18, 2010

(54) Method of administering dose-sparing amounts of formoterol fumarate-budesonide combination particles by inhalation

(76) Inventors: Robert Owen Cook, Hillsborough, NJ (US); Veronica S. De Guzman, Palo Alto, CA (US)

Correspondence Address: Owen J. Bates, Suite 200, 2400 Bayshore Parkway, Mountain View, CA, 94043, US

(21) Appl. No.: 12/800,412

(22) Filed: May 14, 2010

Related U.S. Application Data
(63) Continuation-in-part of application No. 11/988,913, filed on Nov. 23, 2009.
(60) Provisional application No. 61/216,371, filed on May 15, 2009.

(51) Int. Cl. A61K 9/14; A61K 31/58; A61K 9/12; A61P 11/06

(52) U.S. Cl. 424/489; 514/171; 424/43; 424/45

(57) ABSTRACT

Disclosed are methods, and related compositions, that include administering a dose-sparing amount of a formulation that includes inhalation particles to a subject by inhalation; wherein the inhalation particles comprise formoterol fumarate and budesonide, the formoterol fumarate and budesonide being in a distributed encapsulated morphology with respect to one another within said inhalation particles and the formoterol fumarate being in a predetermined mass ratio with regard to the budesonide within said inhalation particles.

### CROSS REFERENCE TO RELATED CASES

This application is a continuation-in-part application under 35 U.S.C. §121 of U.S. application Ser. No. 11/988,913 and furthermore this application claims benefit of U.S. Provisional Application Ser. No. 61/216,371 each of which is incorporated herein by reference in its entirety.

### TECHNICAL FIELD OF THE INVENTION

The invention relates to methods of administering inhalation particles and related formulations in dose-sparing amounts, wherein the inhalation particles comprise formoterol fumarate and budesonide.

### BACKGROUND OF THE INVENTION

The delivery of active pharmaceutical ingredients (APIs) and other therapeutic agents to the respiratory tract via nasal and pulmonary delivery of inhalation particles is widely used for the treatment of a variety of diseases and conditions. Respiratory delivery is accomplished in many ways, such as but not limited to: (i) using an aerosol comprising inhalation particles surrounded by a liquid; (ii) using a multi-dose inhaler; (iii) via the delivery of fine dry powdered inhalation particles via a dry powder inhaler; or (iv) using a nebulizer to nebulize a liquid solution or suspension of the API. The delivery of an API or other therapeutic agents to the respiratory tract offers several advantages, such as, but not limited to, avoidance of metabolism of the drug via the first pass metabolic mechanisms and an increased efficiency of delivery to respiratory tissues (as compared to traditional administration via the bloodstream). Such increased efficiency of delivery to respiratory issues is important in the case of inhaled products that comprise inhaled corticosteroids and long-acting beta agonists.

One example of such products is Symbicort® (inhaled formoterol fumarate & budesonide, available from Astra Zeneca), which is an inhaled product that combines formoterol fumarate and budesonide as a physical mixture of formoterol fumarate-containing particles and budesonide-containing particles.

However, when an admixture of two or more APIs, such as formoterol fumarate and budesonide, is produced by physical blending of the inhalation particles of each API, the ratios/consistency of each drug in the produced particle mixture is not easily controlled and is therefore not reproducible. Further the very process of dispersion into an aerosol can partition such admixture blends of particles by impaction or sedimentation based on the effective aerodynamic diameter of each particle or their agglomerate. For example, if the mass median aerodynamic diameter (MMAD) of a particle of API in the blend is only slightly larger than the MMAD of the other particle of API in the blend, then well known aerodynamic effects will partition out the larger particle API, thereby increasing the fraction of the smaller particle API, in the resulting aerosol, causing a shift from the original fixed combination ratio. A difference in MMAD, say 2.0 microns versus 3.0 microns, at flow rates of 60 liters per minute delivered through the upper respiratory tract of a human could theoretically enrich the small particle API content of the aerosol reaching the lung by approximately 25%. Therefore, the ratio of each drug delivered in a given dose is not consistent and may be considerably different than the intended fixed combination ratio. The inconsistency of the dose could cause serious problems especially when an API is delivered in a much higher or much lower amount than expected. The inhalation delivery of such admixture aerosol particles is inconsistent from dose to dose with the potential to be below therapeutic threshold, or above a safety limit such that adverse events are observed. For example formoterol fumarate, one of the two actives in the Symbicort admixture has a minimum efficacy dose of approximately 4 micrograms in adult asthmatics. At levels above about 12 micrograms systemic b2 agonist effects begin to occur. To overcome dose content inconsistency the label dose of Symbicort is 9 micrograms; this ensures enough drug is always delivered to be effective, but risks triggering adverse effects of systemic b2 agonism (BP elevation, heartrate elevation changes in potassium and glucose serum levels and shakes).

Accordingly, methods and compositions that address the problems noted above and in the art are needed.

### SUMMARY OF THE INVENTION

In an aspect, the invention relates to methods that comprise administering a dose-sparing amount of a formulation comprising inhalation particles to a subject by inhalation; wherein the inhalation particles comprise formoterol fumarate and budesonide, the formoterol fumarate and budesonide being in a distributed encapsulated morphology with respect to one another within said inhalation particles and the formoterol fumarate being in a predetermined mass ratio with regard to the budesonide within said inhalation particles.

### BRIEF DESCRIPTION OF THE DRAWINGS

FIG. 1 shows plasma budesonide concentrations as a function of time.

FIG. 2 shows the study design for Example 2.

FIG. 3 shows the mean plasma budesonide AUC240 characterized by emitted dose, as determined according to Example 2.

### DETAILED DESCRIPTION OF THE INVENTION

### Introduction

The inventors have found, surprisingly, that the problems noted in the art can be addressed by providing methods, along with related compositions, that comprise administering a dose-sparing amount of a formulation comprising inhalation particles to a subject by inhalation; wherein the inhalation particles comprise formoterol fumarate and budesonide, the formoterol fumarate and budesonide being in a distributed encapsulated morphology with respect to one another within said inhalation particles and the formoterol fumarate being in a predetermined mass ratio with regard to the budesonide within said inhalation particles.

The inventors have found that dosing consistency may be increased by combining formoterol fumarate and budesonide into a single particle, minimizing dose partitioning during aerosol generation and during inhalation delivery. Therefore the dose of formoterol can be reduced from that of the Symbicort, 9 micrograms is reduced to 5.4 micrograms, and the dose of the budesonide can be reduced from 80 micrograms to 52 micrograms in the instant invention whilst maintaining equivalent efficacy to Symbicort as measured by the pharmacodynamic standard, FEV1 and by pharmacokinetics. Thus a dose sparing amount is achieved.

Conventional techniques generally produce inhalation particles that contain only one API or inhalation particles that contain a combination of APIs where the APIs are comingled with one another as admixtures or physical blends. As a result, certain useful properties of inhalation particles containing one or more APIs cannot be exploited. For example, it would be beneficial when using a combination of APIs to provide an inhalation particle that contained an essentially pure kernel or central unified portion of a first API that is coated or substantially coated with a second API (of course, the first API could also coat or substantially coat a central unified portion of the second API).

In this manner certain properties of the inhalation particles could be selected based upon the selection of first and second APIs. Such particles are referred to in the art as core/shell, encapsulated or coated. In one embodiment, the second API could protect the first API from degradation or instability by forming a protective coating around the first API. In such a case a first API that was prone to degradation or instability could be protected from such by the second API. In addition a single, discrete inhalation particle comprising two or more APIs would be advantageous in order to control the delivery of the first and/or second APIs or to control the pharmacological availability of the first and/or second APIs. Such a composition of an inhalation particle and formulations comprising such inhalation particles have not been previously known in the art. Furthermore, a single, discrete inhalation particle comprising two or more APIs would be advantageous since the delivery of both drugs would be directed to a single target cell, maximizing the potential synergy of both APIs and controlling the ratio of delivery of each API to a given cell.

Regardless of morphology of the inhalation particles, the presence of the first and the second API in each discrete inhalation particle promotes the coincidental delivery of the first and second API. As used herein, the term “coincidental delivery” means that the first and second APIs are delivered to the same cell at the same time. The coincidental delivery of the first and second APIs offers therapeutic advantages not previously known in the art. Although not wishing to be bound by any particular mechanism, two mechanisms to explain this therapeutic advantage are (1) activation or ‘priming’ of the glucocorticoid receptor (GR) by the beta-agonists making it more receptive to the inhaled corticosteroid and (2) the increased translocation of the inhaled corticosteroid-glucocorticoid receptor complex into the cell nucleus (where the complex exerts biological activity) by the beta-agonists.

For example, when two or more drugs are formulated together such that each drug is present in discrete particles, the delivery of each drug to the same cell and/or the order of delivery cannot be controlled. Therefore, it is difficult to ensure that each cell in need of treatment receives each drug. The inhalation particles of the present disclosure solve this problem. Furthermore, by selecting the desired morphology and the first and second API, not only can the coincidental delivery of the first and second APIs be ensured, the order of release of the first and second APIs can be controlled and determined to achieve maximum therapeutic benefit.

Example 1 illustrates a formulation that could be useful in the practice of the present invention when administered in dose sparing amounts.

Example 2 shows that the recited formulations can be administered in dose sparing amounts. In this example a pharmacodynamic measure, the FEV1 (Forced Expiratory Volume in 1 second), was determined for MAP0005 formulation (2 puffs and 6 puffs) and Symbicort (2 puffs) in a crossover PK/PD trial. The FEV1 response is a direct measure of the clinical efficacy of the formoterol fumarate active contained in both of these combination products. MAP0005 (2 puffs) dose contained 104 micrograms of budesonide combined with 5.4 micrgrams of formoterol fumarate. Symbicort (2 puffs) dose contained 160 micrograms of budesonide and 9.0 micrograms of formoterol fumarate. The FEV1 increase for the MAP0005 (2 puffs) and the Symbicort (2 puffs) doses were comparable, indicating no difference in efficacy despite having MAP0005 (2 puffs) contained only 60% of dose of formoterol fumarate as contained in Symbicort (2 puffs). Further the FEV1 increases compared closely with pivotal clinical trials of Symbicort and Foradil commercial products which contain formoterol furmarate as an active as shown in Table 1 at doses ranging from 9 to 24 micrograms.

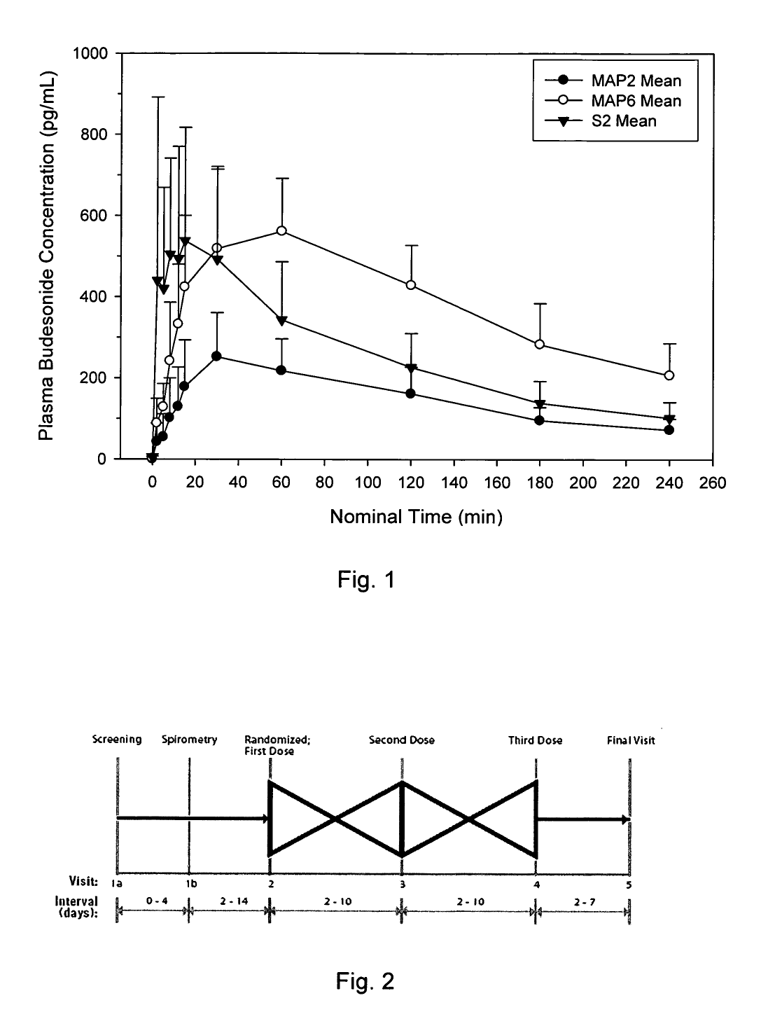

The pharmacokinetic results shown in FIG. 1 demonstrate that the AUCs for budesonide for MAP0005 (2 puffs) MAP2 was reduced proportionally to the dose reduction of approximately 50%. The plasma concentrations of MAP0005 (2 puffs) MAP2 were sufficiently high as to be above known therapeutic threshold for budesonide via inhalation, yet minimized systemic exposure which is undesirable. The Tmax are not clinically different.

This comparison demonstrates that substantially less drug is required when administered according to the present invention than is required using the conventional Symbicort® formulation. Administering less drug is advantageous in reducing adverse events associated with the chronic administration of long-acting beta agonists and steroids.

TABLE 1
Foradil Package Insert
12 ug Formoterol Fumarate
FIG. 1a: First Treatment Day
Increase in Absolute FEV1 from predose baseline

| | Predose | Change at | | Change at | |
|---|---|---|---|---|---|
| | FEV1 | 15 minutes | | 120 minutes | |
| | Baseline (Liters) | Liters | | Liters | |
| 12 ug Formoterol dose (1 puff) | 2.25 | 0.40 | 18% | 0.50 | 22% |
| 24 ug Formoterol dose (2 puffs) | 2.30 | 0.50 | 22% | 0.70 | 30% |

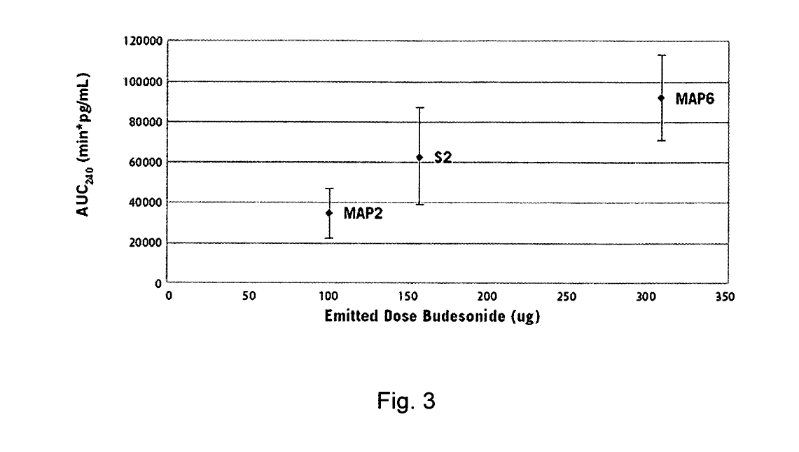

Symbicort Package Insert
Symbicort: 160 ug Budesonide/4.5 ug Formoterol Fumarate vs
4.5 ug Formoterol Fumarate vs
160 ug Budesonide followed by 4.5 ug Formoterol Fumarate
FIG. 3: First Treatment Day
Percentage increase in FEV1 from predose baseline

| | Predose | Change at | | Change at | |
|---|---|---|---|---|---|
| | FEV1 | 15 minutes | | 120 minutes | |
| | Baseline (Liters) | Liters | | Liters | |
| 9 ug Formoterol Fumarate | N/A | N/A | 11% | N/A | 19% |
| (2 puffs Symbicort) | | | | | |
| 320 ug Budesonide plus | N/A | N/A | 12% | N/A | 20% |
| 9 ug Formoterol | | | | | |
| Fumarate (2 puffs) | | | | | |
| 9 ug Formoterol Fumarate | N/A | N/A | 11% | N/A | 18% |
| (2 puffs) | | | | | |

MAP0005 Combi Study
Symbicort: 80 ug Budesonide/4.5 ug Formoterol Fumarate vs
MAP0005: 52 ug Budesonide/2.7 ug Formoterol Fumarate
Increase in Absolute FEV1 from predose baseline

| | Predose | Change at | | Change at | |
|---|---|---|---|---|---|
| | FEV1 | 15 minutes | | 120 minutes | |
| | Baseline (Liters) | Liters | | Liters | |
| 5.4 ug Formoterol Fumarate | 3.41 | 0.38 | 11% | 0.44 | 13% |
| dose (2 puffs MAP0005) | | | | | |
| 16.2 ug Formoterol Fumarate | 3.32 | 0.38 | 12% | 0.57 | 17% |
| dose (6 puffs MAP0005) | | | | | |
| 9 ug Formoterol Fumarate | 3.35 | 0.40 | 12% | 0.52 | 16% |
| dose (2 puffs Symbicort) | | | | | |

The invention will now be described in more detail.

### DEFINITIONS

All publications, patents and patent applications cited herein, whether supra or infra, are hereby incorporated by reference in their entirety for all purposes.

As used herein and in the appended claims, the singular forms “a,” “an,” and “the” include plural reference unless the context clearly dictates otherwise. Thus, for example, a reference to “a particle” includes a plurality of such particles, and a reference to “a carrier” is a reference to one or more carriers and equivalents thereof, and so forth.

“Administering” or “administration” means dosing a pharmacologically active material, such as formoterol fumarate and/or budesonide, to a subject in a manner that is pharmacologically useful.

“Distributed encapsulated” means that the formoterol fumarate is partially encapsulated by the budesonide. Distributed means that the formoterol fumarate exists in a plurality of independent, noninterconnected phase domains distributed within a continuous matrix of budesonide. As used herein, “partially” means that certain domains of the formoterol fumarate are completely encapsulated by the budesonide and certain domains of the formoterol fumarate are exposed on the surface of the inhalation particle. In one example of this embodiment, the formoterol fumarate has a surface area exposed at the surface of the inhalation particle of greater than 10% but less than or equal to 50% of the total exterior surface area of the inhalation particle and the budesonide covers and/or protects from 89.9% to 50% of the formoterol fumarate. In another example of this embodiment, the formoterol fumarate has a surface area exposed at the surface of the inhalation particle of greater than 10% but less than or equal to 90% of the total exterior surface area of the inhalation particle and the budesonide covers and/or protects from 89.9% to 10% of the formoterol fumarate. In yet another example of this embodiment, the formoterol fumarate has a surface area exposed at the surface of the inhalation particle of greater than 10% but less than or equal to 99% of the total exterior surface area of the inhalation particle and the budesonide covers and/or protects from 89.9% to 1% of the formoterol fumarate. Surface coverage or exposure can be measured using exponential dilution titration HLPC spectroscopy. In one example of this embodiment, the formoterol fumarate is present in a volume percentage in the inhalation particles of between 0.1 and 36% by volume.

“Dose-sparing amount” means an amount (e.g. specified number of mass units) of formoterol fumarate and budesonide that provides a desired therapeutic outcome wherein the dose-sparing amount is an amount which is numerically less than would be required to provide substantially the same therapeutic outcome (such as FEV1) if administered separately. In this context, “separately” means that the formoterol fumarate and budesonide are administered in separate particles that are physically blended.

“Formulation” means an inventive composition that comprises inhalation particles and additional pharmaceutically active or inactive ingredients. In embodiments, such additional pharmaceutically active or inactive ingredients may comprise one or more propellants such as hydrofluoralkanes, chlorofluoroalkes, alkanes, carbon dioxide, or blends thereof; a carrier; a stabilizer; an excipient; a preservative; a suspending agent; a chelating agent; a complexing agent; a diluent; a co-solvent or a combination of any of the foregoing.

“Inhalation” means delivery of a drug, such as formoterol fumarate and budesonide particles, to the lung via inhalation through the mouth or nose.

“Inhalation Device” means inhalation particles that comprise formoterol fumarate and budesonide in a device suitable for administration to a subject by inhalation. In embodiments of the present invention, preferred inhalation devices comprise pressurized metered dose inhalers, breath actuated pressurized metered dose inhalers, dry powder inhalers, nebulizers including vibrating mesh, ultrasonic and jet nebulizers, or soft mist inhalers.

“Inhalation particles” means particles that comprises pharmacologically active ingredients and are suitable for administration by inhalation. In an embodiment according to the invention, inhalation particles comprise formoterol fumarate and budesonide.

“Subject” means an animal, including mammals such as humans and primates, that is the object of treatment or observation.

### Formulation and Dosage Forms

Inhalation particles generally useful in the practice of this invention are described in published patent application WO 2007/011989, entitled “Multiple Active Pharmaceutical Ingredients Combined in Discrete Inhalation Particles and Formulations Thereof” by Nahed M. Mohsen, Thomas A. Armer, and Robert 0. Cook. The inhalation particles of the present disclosure may be created using methods including, but are not limited to, the use of supercritical fluid (SCFi) precipitation or sub-supercritical (i.e., near supercritical) precipitation techniques and solution precipitation techniques. Suitable SCF techniques include, as but not limited to, rapid expansion (RES), solution enhanced diffusion (SEDS), gas-anti solvent (GAS), supercritical antisolvent (SAS), precipitation from gas-saturated solution (PGSS), precipitation with compressed antisolvent (PCA), and aerosol solvent extraction system (ASES). The use of SCF processes to form particles is reviewed in Palakodaty, S., et al., “Phase Behavioral Effects on Particle Formation Processes Using Supercritical Fluids”, Pharmaceutical Research, vol. 16. p. 976 (1999). These methods permit the formation of micron and sub-micron sized particles with differing morphologies depending on the method and parameters selected. Suitable SCF and SEDS processes are also described in WO-95/01221, WO-96/00610, WO-98/36825, WO-99/44733, WO-99/52507, WO-99/52550, WO-99/59710, WO-00/30613, WO-00/67892, WO-01/03821, WO-01/15664, WO-02/058674, WO-02/38127, and WO-03/008082. Furthermore the methods described in U.S. patent application Ser. No. 10/264,030 may be used to prepare such inhalation particles. Li addition, the inhalation particles can be fabricated by spray drying, lyophilization, volume exclusion, and any other conventional methods of particle reduction. These methods permit the formation of micron and sub-micron sized particles with differing morphologies depending on the method and parameters selected.

In one particular embodiment, the method used to produce the inhalation particles is a modified ASES system as developed by Eiffel Technologies Limited and as described in a patent application filed on Jul. 15, 2005 and titled “Method of Particle Formation”.

Inhalation particles produced through the use of these methods can be formulated into formulations.

The inhalation particles may be formulated into formulations (such as suspensions) for nebulization by well established methods, such as jet nebulizers, ultrasonic nebulizers, and vibrating orifice nebulizers including Aerogen Aeroneb®, Omron MicroAire®, PARI EFIow™, Boeringher Respimat®, Aradigm AERx®, and next generation nebulizers from Repironics, Ventaira, and Profile Therapeutics. The formulations can be packaged into nebulas by blow/fill/seal technology presented either as a unit container of a biphasic system.

The inhalation particles may also be formulated into aerosol formulations using propellants. Suitable propellants include, but not limited to, hydrofluoroalkanes (HFA) such as the C1-C4 hydrofluorocarbons. Suitable HFA propellants, include but are not limited to, 1,1,1,2,3,3,-heptafluoro-n-propane (HFA 227) and/or 1,1,1,2-tetrafluoroethane (HFA 134) or any mixture of both in any proportions. In one embodiment, the mixture of HFA propellants is selected so that the density of the mixture is matched to the density of the inhalation particles in order to minimize settling or creaming of the inhalation particles. Carbon dioxide and alkanes, such as pentane, isopentane, butane, isobutane, propane and ethane, can also be used as propellants or blended with the C1-4 hydrofluoroalkane propellants discussed above. The formulation may (but is not required to) further comprise carriers, additives and/or diluents as is known in the art.

The inhalation particles produced may be formulated into dry powder formulations, i.e. formulations suitable for use in dry powder inhalation devices. The particles can be used for pulmonary drug delivery by inhalation directly without added carriers, additives or diluents by packaging the inhalation particles into capsules, cartridges, blister packs or reservoirs of a dry powder inhaler (a variety of dry powder inhalers may be used as is known in the art). The inhalation particles may also comprise one or more carriers, additives or diluents to form loose agglomerates of the inhalation particles that are dispersed into individual inhalation particles by the action of the dry powder inhaler. The formulation may (but is not required to) further comprise carriers, additives and/or diluents as is known in the art. Carriers, alone or in combination with other additives, commonly used include, but are not limited to, lactose, dextran, mannitol and glucose. Carriers may be used simply as bulking agents or to improve the dispersibility of the inhalation particles.

If the formulations comprise a carrier, additive or diluent, the total amount of the formoterol fumarate and budesonide is typically about 0.1-99.9% (w/w), about 0.25-75% (w/w), about 0.5-50% (w/w), about 0.75-25% (w/w) or about 1-10% (w/w), based on total weight of the formulation. Such formulations may be prepared by methods known in the art. Formulations as above comprising the inhalation particles described herein may be used for nasal and pulmonary inhalation an appropriate device. As stated above, the formulations may contain added carriers, additives and diluents. The carriers, additives and diluents can be added in the range of 0.0 to 99.9% (w/w) based on the total weight of the formulation. Additives, include, but are not limited to, stabilizers, excipients preservatives, suspending agents, chelating agents, complexing agents and/or other components known to one or ordinary skill in the art. Such carriers, additives and diluents may be a pharmaceutically acceptable grade. Suitable excipients include, but are not limited to ionic and non-ionic surfactants, polymers, natural products and oligomers. Examples of certain suitable excipients that may be used are disclosed in U.S. Pat. Nos. 6,264,739, 5,145,684, 5,565,188 and 5,587,143. In one embodiment, the excipient is an ionic or non-ionic surfactant. Typical surfactants include, but are not limited to, oleates, stearates, myristates, alkylethers, alklyaryl ethers and sorbates and any combination of the foregoing. In a particular embodiment, the surfactant is a polyoxyethylene sorbitan fatty acid ester, such as Tween 20 or Tween 80, sorbitan monooleate (SPAN-80) or isopropyl myristate. Other suitable excipients include polyvinylprrolidine, polyethylene glycol, microcrystalline cellulose, cellulose, cellulose acetate, cyclosdextrin, hydroxypropyl beta cyclodextrin, lecithin, magnesium stearate, lactose, mannitol, trehalose and the like and any combination of the foregoing. The formulations may also comprise polar solvents in small amounts to aid in the solubilization of the surfactants, when used. Suitable polar compounds include C₂₋₆ alcohols and polyols, such as ethanol, isopropanol, polypropylene glycol and any combination of the foregoing. In the event the inhalation particles are to be formulated for use with a dry powder inhaler, lactose, dextran, mannitol and glucose or other suitable compounds may be used. Suitable preservatives, include, but are not limited to, chlorobutanol and benzalkonium chloride and any combination of the foregoing. Suitable chelating agents include, but are not limited to, EDTA and EGTA and any combination of the foregoing. The formulations described above may comprise additional components as well, such as, but not limited to, suspending agents and other components commonly used and known in the art.

It is well known in the art that the size of an inhalation particle determines the depth of penetration into the lung. The depth of penetration is important for achieving the desired therapeutic benefit. In one embodiment, the inhalation particles have a particle size defined as the median mass aerodynamic diameter (MMAD) of less than about 10 microns MMAD, preferably less than about 7.0 microns MMAD, less than about 5.8 microns MMAD, preferably less than about 3 microns in diameter or preferably less than about 1.5 microns MMAD. In certain embodiments, at least 80%, at least 90% or at least 95% of the inhalation particles in a given formulation have an average particle size less than 7.0 microns MMAD. In further embodiments, at least 80%, at least 90% or at least 95% of the inhalation particles in a given formulation have an average particle size less than 5.8 microns MMAD. In one embodiment, the inhalation particles have a particle size greater than about 0.1 microns MMAD, greater than about 1.0 microns MMAD, or greater than about 1.2 microns MMAD, in certain embodiments, at least 80%, at least 90% or at least 95% of the inhalation particles in a given formulation have an average particle size greater than 0.1 microns MMAD. In further embodiments, at least 80%, at least 90% or at least 95% of the inhalation particles in a given formulation have an average particle size less than 1.2 microns MMAD. In embodiments, at least 90% of the inhalation particles have a particle size greater than 0.1 microns MMAD and less than 10 microns MMAD; preferably at least 90% of the particles have a particle size greater than 0.1 microns in diameter and less than 5.8 microns MMAD.

MMAD-is a measure reported in a compendial methods for characterizing aerosol particle size distributions. It is determined by means known and standard in the art such as a cascade impactor, such as an Anderson Cascade Impactor also known as an “Apparatus 1” per USP 601. It is generally known that stages 3-6 detect inhalation particles having a size between about 1.2 and 6.5 microns and that stages 3-8 detect inhalation particles having a size between about 0.26 and 6.5 microns. Inhalation particle sizes between about 1.2 and 6.5 microns or between about 0.26 and 6.5 microns are known as the effective particle size range or the fine particle fraction.

The mass ratio of the formoterol fumarate to the budesonide can be varied. In one embodiment the mass ratio of the formoterol fumarate to budesonide ranges from 50:1 to 1:500. In another embodiment, the mass ratio of the formoterol fumarate to budesonide is from 5:1 to 1:100. In a further embodiment the mass ratio of the formoterol fumarate to budesonide ranges from 1:1 to 1:250. In still another embodiment the mass ratio of the formoterol fumarate to budesonide ranges from 1:1 to 1:80. In another embodiment, the mass ratio of the formoterol fumarate to budesonide ranges from 1:15 to 1:18. In yet another embodiment, the mass ratio of the formoterol fumarate to budesonide is about 1:16.9.

A variety of dosage forms are useful in the practice of the invention, and are described in, for example, US Patent Application Number 2008/0118442. A few embodiments now will be discussed in more detail.

### Dry Powder Inhalers

In a dry powder inhaler (DPI), the dose to be administered is stored in the form of a non-pressurized dry powder and, on actuation of the inhaler, the particles of the powder are inhaled by the subject. Similar to pressurized metered dose inhalers (pMDIs), a compressed gas may be used to dispense the powder. Alternatively, when the DPI is breath-actuated, the powder may be packaged in various forms, such as a loose powder, cake or pressed shape in a reservoir. Examples of these types of DPIs include the Turbohaler™ inhaler (Astrazeneca, Wilmington, Del.) and Clickhaler® inhaler (Innovata, Ruddington, Nottingham, UK). When a doctor blade or shutter slides across the powder, cake or shape, the powder is culled into a flowpath whereby the patient can inhale the powder in a single breath. Other powders are packaged as blisters, gelcaps, tabules, or other preformed vessels that may be pierced, crushed, or otherwise unsealed to release the powder into a flowpath for subsequent inhalation. Typical of these are the Diskus™ inhaler (Glaxo, Greenford, Middlesex, UK), EasyHaler® (Orion, Expoo, FI), and Novohaler™ inhalers. Still others release the powder into a chamber or capsule and use mechanical or electrical agitators to keep the drug suspended for a short period until the patient inhales. Examples of this are the Exubera® inhaler (Pfizer, New York, N.Y.), Qdose inhaler (Microdose, Monmouth Junction, N.J.), and Spiros® inhaler (Dura, San Diego, Calif.).

Pressurized Metered Dose Inhalers pMDIs generally have two components: a canister in which the drug particles are stored under pressure in a suspension or solution form, and a receptacle used to hold and actuate the canister. The canister may contain multiple doses of the formulation, although it is possible to have single dose canisters as well. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the drug particles are atomized, forming an aerosol. pMDIs generally use propellants to pressurize the contents of the canister and to propel the drug particles out of the receptacle outlet. In pMDIs, the composition is provided in liquid form, and resides within the canister along with the propellant. The propellant may take a variety of forms. For example, the propellant may be a compressed gas or a liquefied gas. Chlorofluorocarbons (CFC) were once commonly used as liquid propellants, but have now been banned. They have been replaced by the now widely accepted hydrofluororalkane (HFA) propellants.

In some instances, a manual discharge of aerosolized drug must be coordinated with inhalation, so that the drug particles are entrained within the inspiratory air flow and conveyed to the lungs. In other instances, a breath-actuated trigger, such as that included in the Tempo® inhaler (MAP Pharmaceuticals, Mountain View, Calif.) may be employed that simultaneously discharges a dose of drug upon sensing inhalation, in other words, the device automatically discharges the drug aerosol when the user begins to inhale. These devices are known as breath-actuated pressurized metered dose inhalers (baMDIs).

### Nebulizers

Nebulizers are liquid aerosol generators that convert bulk liquids, usually aqueous-based compositions, into mists or clouds of small droplets, having diameters less than 5 microns mass median aerodynamic diameter (MMAD), which can be inhaled into the lower respiratory tract. This process is called atomization. The bulk liquid contains particles of the therapeutic agent(s) or a solution of the therapeutic agent(s), and any necessary excipients. The droplets carry the therapeutic agent(s) into the nose, upper airways or deep lungs when the aerosol cloud is inhaled.

Pneumatic (jet) nebulizers use a pressurized gas supply as a driving force for liquid atomization. Compressed gas is delivered through a nozzle or jet to create a low pressure field which entrains a surrounding bulk liquid and shears it into a thin film or filaments. The film or filaments are unstable and break up into small droplets that are carried by the compressed gas flow into the inspiratory breath. Baffles inserted into the droplet plume screen out the larger droplets and return them to the bulk liquid reservoir. Examples include PARI LC Plus®, Sprint®, Devilbiss PulmoAide®, and Boehringer Ingelheim Respimat®.

Electromechanical nebulizers use electrically generated mechanical force to atomize liquids. The electromechanical driving force is applied by vibrating the bulk liquid at ultrasonic frequencies, or by forcing the bulk liquid through small holes in a thin film. The forces generate thin liquid films or filament streams which break up into small droplets to form a slow moving aerosol stream which can be entrained in an inspiratory flow.

One form of electromechanical nebulizers are ultrasonic nebulizers, in which the bulk liquid is coupled to a vibrator oscillating at frequencies in the ultrasonic range. The coupling is achieved by placing the liquid in direct contact with the vibrator such as a plate or ring in a holding cup, or by placing large droplets on a solid vibrating projector (a horn). The vibrations generate circular standing films which break up into droplets at their edges to atomize the liquid. Examples include DuroMist®, Drive Medical Beetle Neb®, Octive Tech Densylogic®, and John Bunn Nano-Sonic®.

Another form of an electromechanical nebulizer is a mesh nebulizer, in which the bulk liquid is driven through a mesh or membrane with small holes ranging from 2 to 8 microns in diameter, to generate thin filaments which immediately break up into small droplets. In certain designs, the liquid is forced through the mesh by applying pressure with a solenoid piston driver (AERx®), or by sandwiching the liquid between a piezoelectrically vibrated plate and the mesh, which results in a oscillatory pumping action (EFlow®, AerovectRx, TouchSpray™). In a second type the mesh vibrates back and forth through a standing column of the liquid to pump it through the holes (AeroNeb®). Examples include the AeroNeb Go®, Pro®; PARI EFlow®; Omron 22UE®; and Aradigm AERx®.

Typically, dosage forms according to the invention will be distributed, either to clinics, to physicians or to patients, in an administration kit, and the invention provides such a kit. Such kits comprise one or more of an administration device (e.g., inhalers, etc) and one or a plurality of doses or a reservoir or cache configured to deliver multiple doses of the composition as described above. In one embodiment, the dosage form is loaded with an inventive formulation. The kit can additionally comprise a carrier or diluent, a case, and instructions for employing the appropriate administration device. In some embodiments, an inhaler device is included. In one embodiment of this kit, the inhaler device is loaded with a reservoir containing an inventive formulation. In another embodiment the kit comprises one or more unit doses of the inventive formulation. In one embodiment, the inhaler device is a baMDI such the TEMPO™ Inhaler.

### Methods of Administration

Formulations according to the invention may be administered according to the invention by oral inhalation using inhalation devices such as those discussed elsewhere herein. Dosing frequency may be determined based on the indication being treated and the individual nature of the subject. In embodiments, the inventive inhalation particles or inventive formulations may be administered three times/day, twice/day, or once/day.

In embodiments, dose ranges may comprise:

52 micrograms budesonide/2.7 micrograms formoterol fumarate 2 puffs, QD, BID;
104 micrograms budesonide/5.4 micrograms formoterol fumarate 1 puffs, QD, BID;
104 micrograms budesonide/2.7 micrograms formoterol fumarate 2 puffs, QD or BID; or
208 micrograms budesonide/5.4 micrograms formoterol fumarate 1 puff, QD, BID.

### EXAMPLES

The invention will be more readily understood by reference to the following examples, which are included merely for purposes of illustration of certain aspects and embodiments of the present invention and not as limitations.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein.

Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

### Example 1

### Inhalation Particles and Formulation (Prophetic)

Inhalation particles comprising formoterol fumarate as the formoterol fumarate and budesonide as the budesonide are produced using a modified ASES system as developed by Eiffel Technologies Limited and as described in Australian Patent Application filed on Jul. 15, 2005 and titled “Method of Particle Formation”. The resultant inhalation particles have a formoterol to budesonide mass ratio of 1:16.9. The inhalation particles are in the form of unagglomerated, discrete, fine, white, easily-dispersible powder comprising mainly torroidal-shaped particles of less than 5 micron in diameter when viewed under SEM. In an Aerosizer device that is tested with an Anderson Cascade Impactor with pre-separator and eight stages (refer to Table 1 for the parameters used), the inhalation particles, in their dry powder, neat form have an average emitted dose of 79.2% by mass, an average fine particle fraction of 70.6% by mass (as a percentage of the emitted dose), and an average fine particle fraction of 55.8% by mass (as a percentage of the loaded dose). At least 95% by mass of the fine particle fraction that is deposited on stages 3-6 inclusively, which corresponds to the approximate particle size range 1.2-6.5 micron, and that on each of these stages the formoterol to budesonide mass ratio of the individual inhalation particles is the target ratio of about 1:16.9.

### Example 2

### Clinical Study

This was a randomized, open-label, active-controlled, 3-arm, 3-period, crossover study in adults 18-55 years old, with asthma. Subjects were screened in a two part process and then randomized to receive 3 different treatments (to one of 6 dosing sequences), at 3 dosing periods. Inventive formulations were prepared that had a distributed encapsulated morphology, and a mass ratio of formoterol fumarate dihydrate to budesonide of about 1:16.9. The particles were produced using a supercritical carbon dioxide process as described elsewhere herein.

TABLE 2
Treatment Sequences to Which Subjects Were Randomized

| Sequence | First Dose Period | Second Dose Period | Third Dose Period |
|---|---|---|---|
| A | 2 inhalations invent frmla | 6 inhalations invent frmla | 2 inhalations Symbicort ® |
| B | 6 inhalations invent frmla | 2 inhalations Symbicort ® | 2 inhalations invent frmla |
| C | 2 inhalations Symbicort ® | 2 inhalations invent frmla | 6 inhalations invent frmla |
| D | 2 inhalations invent frmla | 2 inhalations Symbicort ® | 6 inhalations invent frmla |
| E | 6 inhalations invent frmla | 2 inhalations invent frmla | 2 inhalations Symbicort ® |
| F | 2 inhalations Symbicort ® | 6 inhalations invent frmla | 2 inhalations invent frmla |

At each of the 3 dosing periods, blood sampling, vital signs, spirometry, and pulse oximetry were performed immediately before dose administration and at designated times post-dose. Adverse events were monitored throughout the study.

Each subject was medically screened for inclusion (Visit 1 a) and then returned 0 to 4 days later for spirometry and testing for airway reversibility to an inhaled bronchodilator and clinical laboratory investigations (Visit 1 b), prior to being deemed eligible for inclusion in the study. Subjects then returned 2 to 14 days after Visit 1 b and were randomized if still eligible (Visit 2) to one of 6 dosing sequences, received their first treatment, and were observed for a minimum of 4 hours. Subjects returned for their second treatment 2 to 10 days later (Visit 3) and were observed again for a minimum of 4 hours. Subjects returned for their third and final treatment 2 to 10 days later (Visit 4) and were again observed for a minimum of 4 hours. Subjects returned for a final termination visit (Visit 5) 2 to 7 days after receiving their final treatment. The study design is shown generally in FIG. 2.

Oral corticosteroid use was not permitted for 4 weeks prior to and throughout the study. All oral, inhaled, topical (dermal, intranasal or rectal) or systemic glucocorticosteroids that contained the active pharmaceutical ingredient budesonide were excluded at study enrollment and throughout the study duration for any given subject. Inhaled corticosteroid use was not permitted for at least 12 hours, short-acting 112-agonists for 4 hours, oral and long-acting β12-agonists for 12 hours, anticholinergics for 12 hours, and slow release theophyllines for 48 hours prior to spirometry assessment (Visit 1 b) to determine if subjects met inclusion criteria.

Disodium cromoglycate and inhaled corticosteroids use, other than budesonide, were permitted, provided doses remained constant for 4 weeks prior to study, through the end of study. If the subject was taking budesonide at the time of screening, it was substituted with an alternative inhaled corticosteroid for the course of the study as directed by the study investigator. Subjects who had budesonide substituted with an alternative inhaled corticosteroid were required to wait a minimum of 7 days before receiving their first treatment.

### Study Treatments

Inventive formulation using TEMPO® breath-synchronized inhaler (Emitted dose: 52 μg budesonide and 2.7 μg formoterol per inhalation, TEMPO® breath-actuated metered dose inhalers obtained from MAP Pharmaceuticals Inc., Mountain View Calif.)
Symbicort® (inhaled formoterol and budesonide combination therapy) using the Turbuhaler® dry powder inhaler (Emitted dose: 80 μg of budesonide and 4.5 μg of formoterol per inhalation)
Each subject received the following three treatments:
   2 inhalations of inventive formulation using TEMPO®-104 μg budesonide and 5.4 μg formoterol emitted dose
   6 inhalations of inventive formulation using TEMPO®-312 μg budesonide and 16.2 μg formoterol emitted dose
   2 inhalations of Symbicort® (S2)-160 μg budesonide and 9.0 μg formoterol emitted dose

### Study Population

A total of 17 subjects were enrolled in the intent-to-treat population. Of the 17 subjects enrolled, 2 subjects did not complete all 3 treatments. Therefore 15 subjects were available for evaluation in the per-protocol evaluable population for each treatment and available for pharmacodynamic assessments. PK and PD measurements were performed at each treatment visit for each subject.

### Study Inclusion Criteria

Male or female subjects ≧18 and ≦55 years of age with documented and confirmed current history of asthma
FEV1 60-95% of predicted at screening visit 1b and on study confinement days after withholding controller medication (inhaled corticosteroids or anticholinergics for at ≧12 hours, short acting bronchodilators ≧4 hours and long acting bronchodilators ≧12 hours)
≧200 mL or 12% increase of FEV1 after inhalation of 400 μg albuterol after withholding controller medication (inhaled corticosteroids and anticholinergics for ≧12 hours, short acting bronchodilators for 4 hours and long acting bronchodilators ≧12 hours)
Non-smoker (for ≧6 months prior to screening and <10 pack years if previous smoker)
Body mass index ≦30 kg/m2

### Study Exclusion Criteria

Diagnosis of clinically significant COPD, restrictive lung disease, or other pulmonary disease
Had a recent exacerbation of asthma (requiring hospitalization for >1 day or oral prednisolone at ≧30 mg/day for ≧5 days)≦4 weeks prior to screening or a history of life threatening asthma
Abnormal, clinically significant physical or laboratory findings or a medical condition which placed the subject at risk, interfered with the subject's ability to participate in the study, or influenced the safety evaluation
Abnormal 12-lead ECG or rhythm strip (deemed clinically significant by the investigator)
History of significant cardiovascular disease, defined as uncontrolled hypertension, angina pectoris, a history of myocardial infarction, or high blood pressure (≧140/90). Subjects being treated for hypertension, but whose condition was controlled for >3 months, were allowed to enter into the study.

### Study Outcomes

The primary outcome of this study was the plasma budesonide concentration pre- and post-treatment. The pharmacokinetics of budesonide was evaluated for 240 minutes post-dose and the following estimates were performed: The objective of this proof of concept study was to determine the pharmacokinetic profile of the budesonide component of MAP's novel inhaled combination formulation of formoterol particles coated with budesonide, as well as to evaluate the consistency of budesonide delivery in subjects with asthma when administered by the Tempo® inhaler.

Further, this study was designed to observe the pharmacodynamics of the formoterol component, as measured by the bronchodilator effect.

Pharmacokinetic and pharmacodynamic outcomes were compared to those obtained from a dry powder inhaler formulation of a marketed combination product of budesonide and formoterol (Symbicort®; AstraZeneca).
The inter-subject variability (assessed as percent coefficient of variation) was greater for Cmax after 2 inhalations of Symbicort (64%) than after 6 inhalations (28%) or 2 inhalations (38%) of the inventive formulation.

### Results

### Pharmacokinetics

TABLE 3
Pharmacokinetic Results

| Treatment | | Cₘₐₓ (pg/mL) | AUC₂₄₀ (min*pg/mL) |
|---|---|---|---|
| MAP6 | N | 15 | 15 |
| | Mean | 598 | 92274 |
| | SD | 166 | 21324 |
| | CV(%) | 28 | 23 |
| MAP2 | N | 15 | 15 |
| | Mean | 268 | 35804 |
| | SD | 102 | 12262 |
| | CV(%) | 38 | 34 |
| S2 | N | 15 | 15 |
| | Mean | 677 | 62221 |
| | SD | 436 | 24002 |
| | CV(%) | 64 | 39 |

Key: MAP6 - 6 inhalations of inventive formulation using TEMPO ® inhaler (312 μg bud, 16.2 μg form emitted dose); MAP2 - 2 inhalations of inventive formulation using TEMPO ® inhaler (104 μg bud, 5.4 μg form emitted dose); S2 - 2 inhalations of Symbicort ® using Turbuhaler ® DPI (160 μg bud, 9.0 μg form emitted dose)
AUC and Cmax had greater consistency across subjects versus the commercial comparator (AUC CV = 23, 34 and 39%; Cmax CV = 28, 38 and 64% for MAP6, MAP2 and S2, respectively) The mean AUC240 was lower in subjects receiving 2 inhalations of inventive formulation (35804 min*pg/mL) than subjects receiving 6 inhalations of inventive formulation (92274 min*pg/mL) or 2 inhalations of Symbicort ® (62221 min*pg/mL).

No period or sequence effects on pharmacokinetics were observed, therefore treatment effects were grouped and analyzed by treatment. As expected, the mean Cmax for budesonide was lower in subjects receiving 2 inhalations of inventive formulation (268 pg/mL) than in subjects receiving 6 inhalations of inventive formulation, and 2 inhalations of Symbicort® (598 and 677 pg/mL, respectively).

For dose-proportionality, a ratio of 3.0 would be expected for MAP6 inhalations compared to MAP2. Over the 4 hour pharmacokinetic sampling, the observed ratio was 2.63. In post-hoc analysis for AUCinf, the ratio was calculated at 2.85, showing tendency towards dose proportionality.

Further, the ratio of Mean AUC of 2 inhalations of inventive formulation to 2 inhalations of Symbicort approximated the nominal dose ratio of the two products (0.65 for nominal dose ratio versus 0.57 for mean AUC ratio).

FIG. 3 shows the mean plasma budesonide AUC240 by emitted dose (error bars expressed as SD).

Key: MAP6-6 inhalations of inventive formulation using Tempo inhaler (312 μg bud, 16.2 μg form emitted dose) MAP 2-2 inhalations of inventive formulation using Tempo inhaler (104 μg bud, 5.4 μg form emitted dose) S2-2 inhalations of Symbicort using Turbuhaler DPI (160 μg bud, 9.0 μg form emitted dose)

Budesonide AUC was dose proportional for the two doses of the inventive formulation.

### Pharmacodynamics

TABLE 4
Pharmacodynamic Results

| | Measure- | | | |
|---|---|---|---|---|
| Parameter | ment | MAP6 | MAP2 | S2 |
| Maximum % | Mean | 17.31 | 13.10 | 15.70 |
| Change in | SD | 5.82 | 6.78 | 5.97 |
| FEV₁ | % CV | 34 | 52 | 38 |
| | LS Mean | 18.19 | 14.14 | 16.35 |
| | 95% CI | 15.39, 20.99 | 11.34, 16.94 | 13.55, 19.15 |
| Time to Maxi- | Mean | 136.7 | 137.5 | 120.7 |
| mum Change | SD | 61.84 | 85.47 | 68.81 |
| in FEV₁ | % CV | 45 | 62 | 57 |
| (minutes) | LS Mean | 136.7 | 130.7 | 118.6 |
| | 95% CI | 99.1, 174.3 | 93.1, 168.3 | 81.0, 156.2 |

Key: MAP6 - 6 inhalations of inventive formulation using Tempo inhaler (312 μg bud, 16.2 μg form emitted dose); MAP2 - 2 inhalations of inventive formulation using TEMPO ® inhaler (104 μg bud, 5.4 μg form emitted dose); S2 - 2 inhalations of Symbicort ® using Turbuhaler ® DPI (160 μg bud, 9.0 μg form emitted dose).
Mean maximum % change in FEV1 was >12% for all three treatments, showing clinically significant bronchodilation in asthmatic adults, despite background maintenance therapy. All treatments exceeded a mean 0.2 L maximal FEV1 change from baseline with no clinically meaningful differences between inventive formulation and Symbicort ®. The mean time to maximum change from baseline in FEV1 was relatively similar among subjects receiving 6 or 2 inhalations of inventive formulation or 2 inhalations of Symbicort ® (136.7, 137.5 and 120.7 min, respectively).

### Safety

No SAEs were reported for the inventive formulation or comparator, and no clinically significant changes in serum potassium were observed.

What is claimed is:

1. A method comprising:
administering a dose-sparing amount of a formulation comprising inhalation particles to a subject by inhalation;
wherein the inhalation particles comprise formoterol fumarate and budesonide, the formoterol fumarate and budesonide being in a distributed encapsulated morphology with respect to one another within said inhalation particles and the formoterol fumarate being in a predetermined mass ratio with regard to the budesonide within said inhalation particles.

2. The method of claim 1 where the formoterol fumarate has a surface area exposed on the surface of the particle of greater than 10% but less than or equal to 90% of the total exterior surface area of the particle.

3. The method of claim 1 where the budesonide covers from 89.9% to 10% of the formoterol fumarate.

4. The method of claim 1 where the formoterol fumarate is present in a mass ratio to the budesonide ranging from 5:1 to 1:100.

5. The method of claim 4 where said ratio ranges from about 1:15 to about 1:18.

6. The method of claim 1 where at least 90% of the particles have a median mass aerodynamic diameter greater than 0.1 microns in diameter and less than 10 microns in diameter.

7. The method of claim 5 where at least 90% of the particles have a median mass aerodynamic diameter greater than 0.1 microns in diameter and less than 5.8 microns in diameter.

8. The method of claim 1 wherein said formulation comprises one or more propellants comprising hydrofluoralkanes, chlorofluoroalkes, alkanes, carbon dioxide, or blends thereof.

9. The method of claim 1 further comprising a carrier, a stabilizer, an excipient, a preservative, a suspending agent, a chelating agent, a complexing agent, a diluent, a co-solvent or a combination of any of the foregoing.

10. The method of claim 1 where said formulation is a pressurized metered dose inhaler formulation.

11. The method of claim 1, further comprising:
providing an inhalation device that administers a dose-sparing amount of the formulation to a subject.

12. The method of claim 11, wherein the inhalation device comprises a pressurized metered dose inhaler, breath actuated pressurized metered dose inhaler, dry powder inhaler, vibrating mesh nebulizer, ultrasonic nebulizer, jet nebulizer, or soft mist inhaler.

13. The method of claim 11 wherein the formoterol fumarate has a surface area exposed on the surface of the particle of greater than 10% but less than or equal to 90% of the total exterior surface area of the particle.

14. The method of claim 11 wherein the budesonide covers from 89.9% to 10% of the formoterol fumarate.

15. The method of claim 11 wherein the formoterol fumarate is present in a mass ratio to the budesonide ranging from 5:1 to 1:100.

16. The method of claim 15 wherein said ratio ranges from about 1:15 to about 1:18.

17. The method of claim 11 wherein at least 90% of the particles have a median mass aerodynamic diameter greater than 0.1 microns in diameter and less than 10 microns in diameter.

18. The method of claim 17 wherein at least 90% of the particles have a median mass aerodynamic diameter greater than 0.1 microns in diameter and less than 5.8 microns in diameter.

19. The method of claim 11 wherein said formulation comprises one or more propellants comprising hydrofluoralkanes, chlorofluoroalkes, alkanes, carbon dioxide, or blends thereof.

20. The method of claim 11 further comprising a carrier, a stabilizer, an excipient, a preservative, a suspending agent, a chelating agent, a complexing agent, a diluent, a co-solvent or a combination of any of the foregoing.

21. The method of claim 11 where said formulation is a pressurized metered dose inhaler formulation.
